(19) United States
(12) Patent Application Publication
(10) Pub. No.: US 2020/0367791 A1
(43) Pub. Date: Nov. 26, 2020

(54) Ground-Truth Data Creation Support System and Ground-Truth Data Creation Support Method

(71) Applicant: Hitachi, Ltd., Tokyo (JP)

(72) Inventors: Shunsuke MINUSA, Tokyo (JP); Takeshi TANAKA, Tokyo (JP); Hiroyuki KURIYAMA, Tokyo (JP); Kazuhito SUGIYAMA, Tokyo (JP)

(21) Appl. No.: 16/824,068

(22) Filed: Mar. 19, 2020

(30) Foreign Application Priority Data
May 24, 2019 (JP) 2019-097854

(51) Int. Cl. A61B 5/11; A61B 5/00; G01P 13/00

(52) U.S. Cl. CPC A61B 5/1118; A61B 5/7275; A61B 2562/0219; A61B 5/1123; G01P 13/00; A61B 5/7267

(57) ABSTRACT

A ground-truth data creation support system comprising: an input unit configured to input sensor data obtained by measurement with a sensor; a storage unit configured to store a code assigning model to assign codes associated with characteristics of sensor data to the sensor data and an activity inferring model to infer an activity of a person wearing the sensor based on the sensor data that has been assigned codes; a processing unit configured to infer an activity in a specific measurement time period of a person wearing the sensor, based on sensor data in the specific measurement time period, the code assigning model, and the activity inferring model; and an output unit configured to output the inferred activity in the specific measurement time period and codes assigned to the sensor data in the specific measurement time period.

### CLAIM OF PRIORITY

The present application claims priority from Japanese patent application JP2019-97854 filed on May 24, 2019, the content of which is hereby incorporated by reference into this application.

### BACKGROUND OF THE INVENTION

This invention relates to technology to support creation of ground-truth data about an activity with sensor data acquired by recording human activities.

To obtain ground-truth data necessary to recognize activities of the user with sensor data such as acceleration data measured by a wearable device, there is proposed a means to generate ground-truth data with sensor data only or to support such generation of ground-truth data. For example, WO 2010/032579 A (Patent Document 1) discloses a system for generating a history of activities that extracts a scene from activity states of a person, identifies activity details for each scene, estimates activity details from the appearance order of the activity details, and present the activity details to the user.

### SUMMARY OF THE INVENTION

The information presented to the user by the system disclosed in Patent Document 1 is merely a feature value such as a level of exertion that is calculated from sensor data in accordance with rules and an recognized activity (such as walking, resting, or light work); it is not enough for the user to determine and input ground-truth data. For this reason, the information to create ground-truth data only from sensor data is mostly based on the user's memory; the accuracy of ground-truth data is not assured.

This invention is achieved in view of the above-described problem, aiming to support creation of accurate ground-truth data about an activity with sensor data in which human activities are recorded.

In order to solve at least one of the foregoing problems, there is provided a ground-truth data creation support system comprising: an input unit configured to input sensor data obtained by measurement with a sensor; a storage unit configured to store a code assigning model to assign codes associated with characteristics of sensor data to the sensor data and an activity inferring model to infer an activity of a person wearing the sensor based on the sensor data that has been assigned codes; a processing unit configured to infer an activity in a specific measurement time period of a person wearing the sensor, based on sensor data in the specific measurement time period, the code assigning model, and the activity inferring model; and an output unit configured to output the inferred activity in the specific measurement time period and codes assigned to the sensor data in the specific measurement time period.

An aspect of this invention provides support in creating accurate ground-truth data about an activity in a specific measurement time period based on sensor data that has been assigned codes and an inference result about the activity. Problems, configurations, and effects other than those described above are clarified in the following description of the embodiments.

### BRIEF DESCRIPTION OF THE DRAWINGS

FIG. 1 is a block diagram illustrating a major configuration of embodiments of this invention.

FIG. 2 is a hardware configuration diagram illustrating a major configuration of a ground-truth data creation support system in Embodiment 1 of this invention.

FIG. 3A is an explanatory diagram of a typical procedure of generating a unit activity model, which is executed by a server in Embodiment 1 of this invention.

FIG. 3B is an explanatory diagram of a typical procedure of generating a working activity model, which is executed by a server in Embodiment 1 of this invention.

FIG. 4 is an explanatory diagram of a typical procedure of generating ground-truth data with the ground-truth data creation support system in Embodiment 1 of this invention.

FIG. 5 is a schematic diagram illustrating data forms in obtaining ground-truth candidate data from sensor data using the ground-truth data creation support system in Embodiment 1 of this invention.

FIG. 6 is an explanatory diagram illustrating an example of an input screen to receive confirmation of a ground-truth from the operator with the ground-truth data creation support system in Embodiment 1 of this invention.

FIG. 7A is an explanatory diagram of a typical example of data structure of unit activity series data stored in the ground-truth data creation support system in Embodiment 1 of this invention.

FIG. 7B is an explanatory diagram of a typical example of data structure of ground-truth candidate data stored in the ground-truth data creation support system in Embodiment 1 of this invention.

FIG. 7C is an explanatory diagram of a typical example of data structure of working activity ground-truth data stored in the ground-truth data creation support system in Embodiment 1 of this invention.

FIG. 7D is an explanatory diagram of a typical example of data structure of learning range data stored in the ground-truth data creation support system in Embodiment 1 of this invention.

FIG. 8A is an explanatory diagram of a typical example of data structure of user data held by the ground-truth data creation support system in Embodiment 1 of this invention.

FIG. 8B is an explanatory diagram of a typical example of data structure of model configuration data held by the ground-truth data creation support system in Embodiment 1 of this invention.

FIG. 9 is a hardware configuration diagram of a ground-truth data creation support system in Embodiment 2 of this invention.

FIG. 10 is a hardware configuration diagram illustrating a major configuration of a ground-truth data creation support system in Embodiment 3 of this invention.

FIG. 11 is an example of an input screen to receive confirmation of a ground-truth from the operator with the ground-truth data creation support system in Embodiment 3 of this invention.

### DETAILED DESCRIPTION OF THE EMBODIMENTS

Hereinafter, embodiments of this invention are described with reference to the drawings.

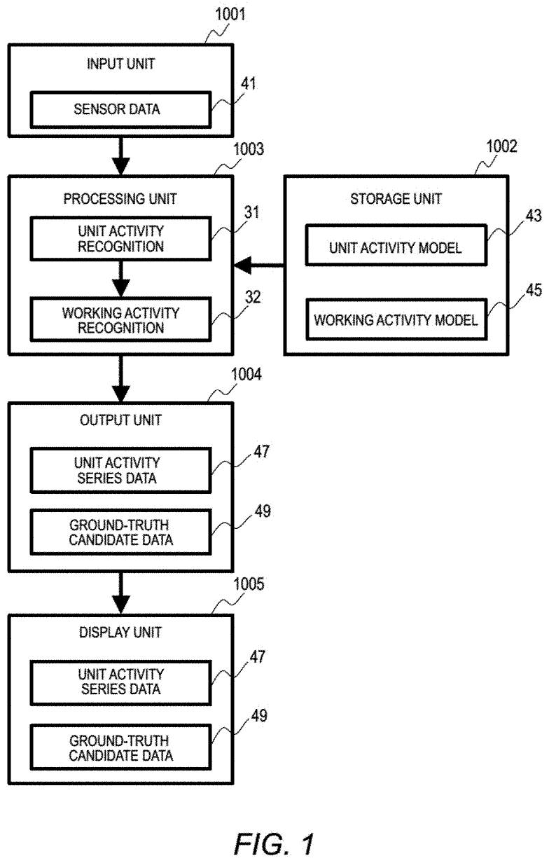

FIG. 1 is a block diagram illustrating a major configuration of embodiments of this invention.

In the ground-truth data creation support systems in the embodiments, the input unit 1001 inputs sensor data 41 obtained by measurement with a sensor to the processing unit 1003. The storage unit 1002 stores a code assigning model (hereinafter, also referred to as unit activity model) 43 and an activity inferring model (hereinafter, also referred to as working activity model) 45. The code assigning model 43 assigns codes associated with characteristics of sensor data corresponding to a plurality of known activity patterns to the sensor data 41. The activity inferring model 45 infers the activity in a specific measurement time period based on the sensor data that has assigned codes (hereinafter, also referred to as unit activity series data) 47.

The processing unit 1003 performs unit activity recognition 31 that generates unit activity series data 47 based on sensor data 41 input from the input unit 1001 and the unit activity model 43 retrieved from the storage unit 1002. The processing unit 1003 subsequently performs working activity recognition 32 that generates an activity (hereinafter, also referred to as ground-truth candidate data) 49 in a specific measurement time period based on the unit activity series data 47 and the working activity model 45 retrieved from the storage unit 1002.

The output unit 1004 outputs the unit activity series data 47 and the ground-truth candidate data 49 generated by the processing unit 1003. The display unit 1005 displays the unit activity series data 47 and the ground-truth candidate data 49 output by the output unit 1004.

This system configuration and the processing of each unit are described using the following embodiments provided with specific hardware configurations.

### Embodiment 1

Embodiment 1 of this invention is described.

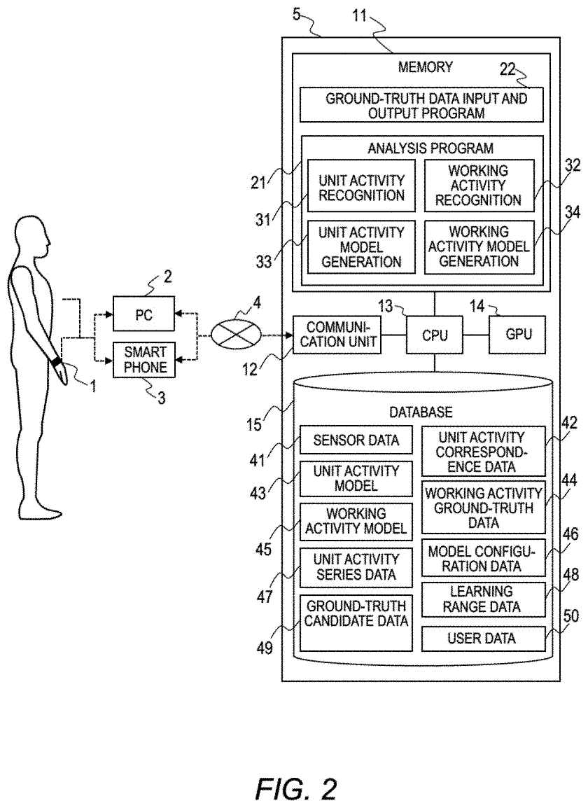

FIG. 2 is a hardware configuration diagram illustrating a major configuration of a ground-truth data creation support system in Embodiment 1 of this invention.

The ground-truth data creation support system in this embodiment includes a sensor 1 to be worn by the user, a PC 2 or a smartphone 3 capable of communicating with the sensor 1, and a server 5 capable of communicating with the PC 2 or smartphone 3 via a network 4. The sensor 1 sends measured sensor data 41 to the server 5 through the PC 2 or smartphone 3.

The server 5 analyzes the received sensor data 41 to calculate unit activity series data 47 and ground-truth candidate data 49. The unit activity series data 47 is time-series data of activity patterns (namely, unit activities) obtained by classifying the sensor data 41 segmented by a short period (for example, six seconds) into characteristic patterns of typical human motions or positions. The ground-truth candidate data 49 is time-series data obtained by inferring activities (working activities) to be recognized by this system from the unit activity series data 47.

The PC 2 or smartphone 3 can download an analysis result, namely unit activity series data 47 and ground-truth candidate data 49, from the server 5 and display them for the user. Furthermore, the PC 2 or smartphone 3 can record whether the displayed ground-truth candidate data 49 is correct to the working activity ground-truth data 44 and further, if the displayed data 49 is wrong, collect the name of the truly correct working activity and the time period from the user and record them to the working activity ground-truth data 44.

For convenience of explanation, this embodiment employs a wristband type of wearable sensor to be attached on a wrist as the sensor 1 and describes an example of processing that supports creation of ground-truth data about working activities with only the sensor data 41 acquired from the sensor 1. Furthermore, the sensor data 41 in the following description is three kinds of acceleration data measured along three axes orthogonal to one another.

In addition to or in place of the acceleration data, time-series data on angular velocity, illuminance, sound, the IDs of sensors in the proximity can be used as sensor data 41. The sensor 1 can be attached to a part other than a wrist, for example, an arm or the waist. The sensor data 41 is sent to the PC 2 or smartphone 3 automatically at the time when wired or wireless connection to the PC 2 or smartphone 3 is established in the network 4 or at a desirable time for the user.

The PC 2 and the smartphone 3 can communicate with not only the sensor 1 but alto the server 5 connected with the network 4 such as the Internet. The PC 2 and the smartphone 3 can send sensor data 41 received from the sensor 1 to the server 5 and further, display and operate data stored in the server 5 and input data to the server 5 with a ground-truth data input and output program 22 in the server 5.

The server 5 includes a communication unit 12, a central processing unit (CPU) 13, a graphics processing unit (GPU) 14, a memory 11, and a database 15. The memory 11 stores a ground-truth data input and output program 22 and an analysis program 21. The server 5 analyzes sensor data 41 sent from the PC 2 or smartphone 3 with the analysis program 21, calculates unit activity series data 47 and ground-truth candidate data 49, and records them to the database 15. The server 5 can also generate a unit activity model 43 and a working activity model 45, which are algorithms or rules to calculate unit activity series data 47 and ground-truth candidate data 49 from sensor data 41.

The CPU 13 performs processing of the analysis program 21 and the ground-truth data input and output program 22. The GPU 14 can cooperate with the CPU 13 in the processing as necessary. In the following, an example where the CPU 13 and the GPU 14 perform the processing of each function in unit activity recognition 31 and the CPU 13 performs the processing of each function in working activity recognition 32 is described. Each function will be described later.

The communication unit 12 connects to the PC 2 or smartphone 3 via the network 4 to send and receive data. The sensor data 41 received from the sensor 1 and the working activity ground-truth data 44 input through the PC 2 or smartphone 3 are recorded to the database 15.

The ground-truth data input and output program 22 is a program for making the CPU 13 perform processing to display data recorded in the database 15 for the user via the network 4 and also, processing to accept input from the user. The analysis program 21 is composed of a unit activity recognition program 31, a working activity recognition program 32, a unit activity model generation program 33, and a working activity model generation program 34.

The database 15 includes sensor data 41, a unit activity model 43, a working activity model 45, unit activity series data 47, ground-truth candidate data 49, unit activity correspondence data 42, working activity ground-truth data 44, model configuration data 46, leaning range data 48, and user data 50.

The unit activity recognition program 31 is a program for making the CPU 13 perform processing of converting received sensor data 41, calculating feature values related to characteristic patterns of the user's typical motions and positions, grouping the calculated feature values to analogous feature value groups (unit activities), and recording the assigned feature value group identifiers (unit activity IDs) to unit activity series data 47, with a unit activity model 43. The working activity recognition program 32 is a program for making the CPU 13 perform processing of converting the unit activity series data 47, inferring a working activity of the user, and recording the inferred working activity to the ground-truth candidate data 49, with a working activity model 45. The unit activity model generation program 33 is a program for making the CPU 13 perform processing of generating a unit activity model 43 based on the sensor data 41, the learning range data 48, and the unit activity correspondence data 42. The working activity model generation program 34 is a program for making the CPU 13 perform processing of generating a working activity model based on unit activity series data 47 specified in the learning range data 48 and the working activity ground-truth data 44. Each program can be executed either at a time desired by the user or in response to a trigger of data input from the sensor 1.

The illustrated in FIG. 2 is an example of hardware configuration for implementing the ground-truth data creation support system in FIG. 1. For example, the function of the input unit 1001 in FIG. 1 can be implemented by the CPU 13 inputting the sensor data 41 received from the sensor 1 via the PC 2 or smartphone 3, the network 4, and the communication unit 12 to a process of the analysis program 21. The function of the input unit 1001 can also be implemented by the CPU 13 retrieving the sensor data 41 stored in the database 15 and inputting the sensor data 41 to a process of the analysis program 21. In addition, when the PC 2 or smartphone 3 receives input of information from the user, the information is likewise input to a process executed by the CPU 13 through the network 4 and the communication unit 12. In other words, the function of the input unit 1001 can be considered as a function of the CPU 13 or a function of the CPU 13, the communication unit 12, and the PC 2 or smartphone 3.

The function of the processing unit 1003 can be implemented by the CPU 13 executing a program (for example, the analysis program 21) stored in the memory 11, for example. The storage unit 1002 can be implemented by a storage device such as an HDD or a flash memory, for example, which corresponds to the database 15 in FIG. 2.

The function of the output unit 1004 can be implemented by the CPU 13 executing a program (for example, the ground-truth data input and output program 22) stored in the memory 11, for example.

The function of the display unit 1005 can be implemented by a display device (not shown) of the server 5, for example. The function of the display unit 1005 can also be implemented by the PC 2 or smartphone 3. In this case, the data output by the output unit 1004 is sent to the PC 2 or smartphone 3 through the communication unit 12 and the network 4 and the PC 2 or smartphone 3 displays an image based on the data on its display device (not shown).

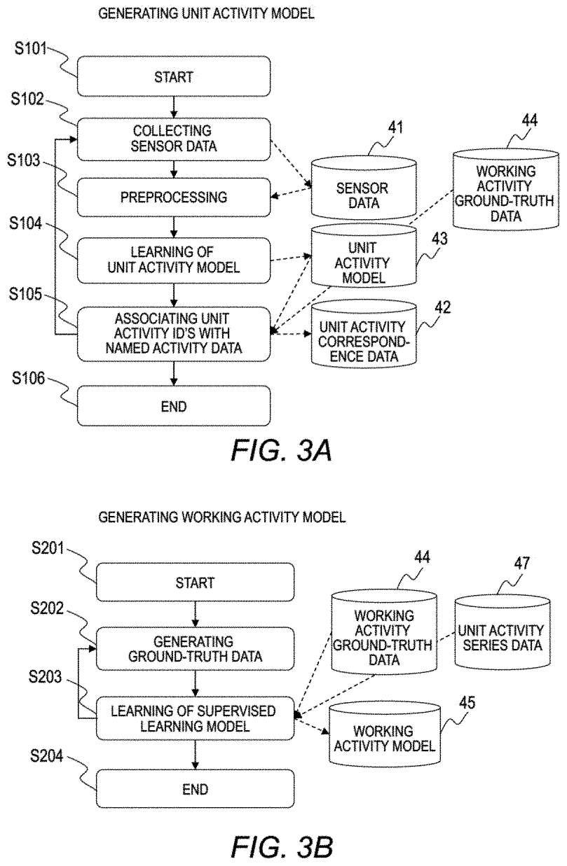

FIG. 3A is an explanatory diagram of a typical procedure of generating a unit activity model S101, which is executed by the server 5 in Embodiment 1 of this invention.

FIG. 3B is an explanatory diagram of a typical procedure of generating a working activity model S201, which is executed by the server 5 in Embodiment 1 of this invention.

Generating a unit activity model S101 (FIG. 3A) includes collecting sensor data S102, preprocessing S103, learning of a unit activity model S104, and associating unit activity IDs with named activity data S105.

In the collecting sensor data S102, the server 5 receives sensor data 41 from the sensor 1 attached on the user.

Next, preprocessing S103 is performed. For example, the sensor data 41 can be adjusted in orientation in accordance with the attachment position of the sensor 1 because the sensor data 41 collected by the sensor 1 has different information depending on the attachment position to the user or orientation of the sensor 1. In addition, removal of the gravitational component from the sensor data 41 to focus attention particularly on motion and normalization to reduce the differences in intensity of motion among users can also be employed. Thereafter, the sensor data 41 is segmented by a predetermined time unit (window width).

The sensor data 41 preprocessed in S103 is input to learning of a unit activity model S104. The learning of a unit activity model S104 is not supervised learning but unsupervised learning that extracts feature values related to characteristic patterns such as typical human motions and positions that are useful to recognize activities from the sensor data 41, groups the sensor data 41 into unit activities analogous in feature values, and assigns unit activity IDs.

Typically, the learning of a unit activity model S104 is unsupervised learning that successively executes a known feature extraction calculation and a known clustering calculation so that the unit activities obtained by the generated unit activity model 43 will be feature value groups that are easy for humans to interpret. In order to eliminate the necessity to manually define characteristic activities but extract probable features, the unit activity model 43 can employ a model utilizing an autoencoder for feature extraction and a k-means for clustering to assign cluster identifiers as unit activity IDs to the input sensor data 41. Another example can employ a machine learning algorithm that repeats feature extraction and clustering for a plurality of times to obtain well-separated clusters.

In preparing the unit activity model 43, it is preferable to prepare not only a single unit activity model 43 but also a plurality of models different in hyperparameters such as the window width for the sensor data 41 to be input and the number of clusters to be obtained by the unit activity recognition so that the model to be used to generate unit activity series data is selectable in accordance with the demand of the user.

Since the unit activity model 43 defined by feature extraction and clustering calculation is a classification algorithm obtained by unsupervised learning, it is unnecessary to manually define basic activities such as typical motions and positions. Unit activities can be obtained by specifying the number of unit activities to be extracted. However, this configuration changes the unit activities meant by individual unit activity IDs each time the unit activity model 43 is revised. To eliminate this problem and define the unit activities by feature value groups that are easy for humans to interpret, unit activity correspondence data 42 is used.

The unit activity correspondence data 42 typically includes an identifier uniquely identifying sensor data 41 (a known activity pattern) to be an input to the unit activity model 43, an activity pattern name (for example, slow movement) that is the unit activity name for the sensor data 41 or the name of an activity pattern associated with the features of the sensor data 41, and a unit activity ID that is a code assigned to the sensor data. Recording unit activity IDs determined as a result of inputting sensor data 41 recorded in the unit activity correspondence data 42 to a newly generated unit activity model 43 to the unit activity correspondence data 42 (S105) enables humans to easily understand the correspondence of the characteristic pattern meant by a unit activity, even in the case where the unit activity model 43 is revised.

Ground-truth data of the working activity model 45 and ground-truth data collected by the ground-truth data input and output program 22, which will be described later, can be reused to associate unit activity IDs with sensor data 41. Instead of generating the unit activity correspondence data 42, interpreting the meanings of the characteristic patterns can be performed later based on examples of sensor data included in the individual clusters assigned unit activity IDs.

This is the end of the generation of a unit activity model S101 (S106). The generation of a unit activity model S101 can be executed automatically upon receipt of sensor data 41 from the sensor 1, periodically, or at any time as desired by the user.

For example, some condition can be determined in advance and when this condition is satisfied, the server 5 can execute the process to generate a unit activity model S101 and updates the unit activity model 43 in accordance with the result. Examples of the condition include that a specific amount of new sensor data 41 is input and that sensor data 41 about a new user is input. This new user can be a user belonging to a new field.

Using more sensor data 41, sensor data 41 about more users, or sensor data 41 about users in more fields leads to generation of a more accurate unit activity model 43.

Generating a working activity model S201 (FIG. 3B) includes generating ground-truth data S202 and learning of a supervised learning model S203.

The generating ground-truth S202 is different between the first processing to generate a working activity model 45 and the second and the subsequent processing.

To generate a working activity model 45 for the first time, ground-truth data has to be generated by some means. A known method can be employed for this means: recording the user's activities through visual surveillance, taking out activities recorded in motion pictures or a video footage, or recording the user's activities by himself or herself can be employed.

In generating a working activity model 45 for the second and subsequent times, ground-truth data is generated using the ground-truth data input and output program 22, which will be described later in this Embodiment 1. Typically, a record of ground-truth data generated somehow includes an applied sensor data range, information on the unit activity model 43 used in generating the input unit activity series data 47, and the working activity name of a ground-truth; it is recorded to the working activity ground-truth data 44.

After generating the ground-truth data to be used to generate a working activity model 45, the server 5 executes learning of a supervised learning model S203. The input for the working activity model 45 is unit activity series data 47 segmented by a predetermined time unit (window width) and working activity ground-truth data 44 therefor. Since the unit activity series data 47 is time-series unit activity IDs in numerical values or symbols, a known supervised learning model capable of handling discrete data or symbol strings is selected for the working activity model 45.

An example of a working activity model 45 that uses the frequencies of unit activities included in a predetermined time unit is a model that first converts the frequencies of unit activities by latent Dirichlet allocation, which is a method of topic analysis used in document analysis, into topic probabilities that can be easily interpreted by humans and subsequently uses gradient boosting, which is an ensemble learning method having high recognition performance. Another example of the working activity model 45 that uses the time series of unit activities is a model utilizing a recurrent neural network with long short-term memory.

In preparing the working activity model 45, it is also preferable to prepare not only a single working activity model 45 but also a plurality of models different in hyperparameters such as the window width for the sensor data 41 to be input and the number of clusters obtained by the unit activity recognition so that the model to be used to generate ground-truth candidate data 49 is selectable in accordance with the demand of the user.

The aforementioned sensor data 41 recorded in the unit activity correspondence data 42 can be used as ground-truth data for the working activity model 45.

The above-described processing to generate a working activity model S201 can be executed automatically upon receipt of sensor data 41 from the sensor 1, periodically, or at any time as desired by the user.

The unit activities to be recognized by the unit activity model 43 are comparatively generalized irrespective of the applied field such as nursing care field; however, the working activities to be recognized by the working activity model 45 can be significantly different among applied fields. Accordingly, it is expected that the frequency of generating a unit activity model 43 is less than the frequency of generating a working activity model 45.

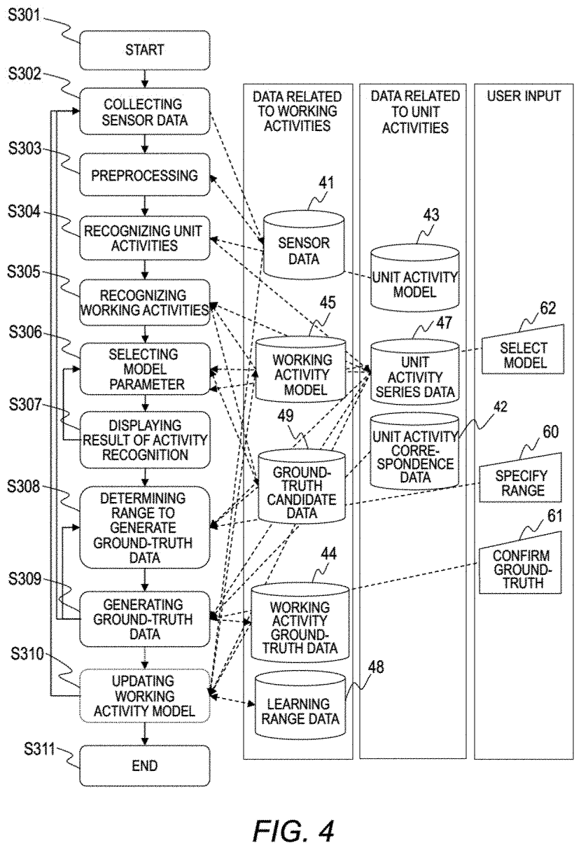

FIG. 4 is an explanatory diagram of a typical procedure of generating ground-truth data S301 with the ground-truth data creation support system in Embodiment 1 of this invention.

The generating ground-truth data S301 with the ground-truth data creation support system in this embodiment typically includes collecting sensor data S302, preprocessing S303, recognizing unit activities S304, recognizing working activities S305, selecting the model parameter S306, displaying a result of activity recognition S307, determining a range to generate ground-truth data S308, generating ground-truth data S309, and updating a working activity model S310.

In the collecting sensor data S302, the server 5 receives sensor data collected by the sensor 1 attached on the user and records it as sensor data 41, as described in the foregoing description of FIG. 3A. In the subsequent preprocessing S303, adjustment in orientation or position, removal of the gravitational component, and/or normalization are performed on the sensor data 41 and further, the sensor data 41 is segmented by a predetermined window width, as described above.

The preprocessed sensor data is converted to unit activity series data 47 with the unit activity model 43 (S304). Typically, the obtained unit activity series data 47 is stored as records each including a time, a unit activity ID at the time, information on the unit activity model 43 used in the conversion, and user information.

In this processing, it is preferable to convert the sensor data 41 with a plurality of unit activity models 43 different in hyperparameter such as the window width for the sensor data 41 and store each result as unit activity series data 47. Then, the ground-truth data input and output program 22 can display the unit activity series data 47 in pseudo real-time without recalculation in displaying the unit activity series data 47 obtained by unit activity models 43 different in hyperparameter.

The description hereinafter is provided based on an assumption that the unit activity model 43 includes the window width for the sensor data 41 to be input as a hyperparameter and unit activity series data 47 obtained by a plurality of unit activity models 43 different in value of the window width is stored.

Next, the unit activity series data 47 is segmented again by a predetermined window width and converted to ground-truth candidate data 49 with the working activity model 45 (S305). Typically, the obtained ground-truth candidate data 49 is stored as records each including a time, probabilities (probabilities of works) that the input unit activity series data 47 belongs to individual working activities to be recognized at the time, a working activity at this time, information on the unit activity model used to calculate the input unit activity series data 47, information on the working activity model 45 used in the conversion, and user information.

Regarding the data structure of unit activity series data 47, records each including a time period in which a working activity is continued, the working activity in this time period, information on the unit activity model used to calculate the input unit activity series data 47, information on the working activity model 45 used in the conversion, and user information can be stored.

Like the unit activity series data 47, it is preferable to store ground-truth candidate data 49 obtained from a plurality of sets of unit activity series data 47 differing in hyperparameter and ground-truth candidate data 49 converted by a plurality of working activity models 45 different in hyperparameter as ground-truth candidate data 49. The description hereinafter is provided based on an assumption that the working activity model 45 is applied to a plurality of sets of unit activity series data 47 differing in hyperparameter and ground-truth candidate data 49 in different window widths is obtained.

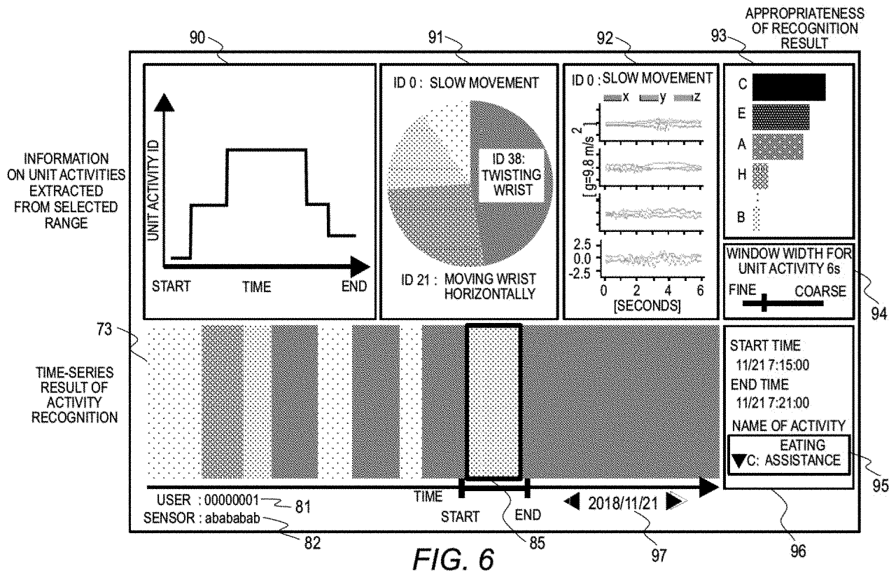

Subsequently, selecting the model parameter S306 and displaying a result of activity recognition S307 with the ground-truth data input and output program 22 are performed. In these phases, the user operates the PC 2 or smartphone 3 to perform model selection 62 by selecting a desired window width as the model parameter of the unit activity model 43, with a knob (see the region 94 in FIG. 6), for example. The ground-truth data input and output program 22 retrieves unit activity series data 47 and ground-truth candidate data 49 in accordance with the input selection 62 and displays a result of activity recognition like the display example display (see FIG. 6) on the PC 2 or smartphone 3. The example of display will be described later.

Selecting the model parameter S306 and displaying a result of activity recognition S307 can be repeated for a plurality of times until the user obtains a desired result. Although the window width of the unit activity model 43 is selected as the model parameter in this example, the ground-truth data input and output program 22 can be configured to accept input of model selection 62 for each of the unit activity model 43 and the working activity model 45 to determine their hyperparameters, if the unit activity model 43 and the working activity model 45 have hyperparameters different from window width. The actually used hyperparameters are recorded to the user data 50 to be used in analyzing hyperparameters suitable for the applied field.

Depending on the applied field (such as nursing care or construction), characteristics of users' activities could be different and as a result, the parameter values suitable for the model to recognize an activity could be different. However, an appropriate parameter can be determined to generate an accurate model by repeating the processing while changing the parameter as described above. As already described, processing in pseudo real-time that instantly displays a result in response to input from the user is also available by calculating results in advance with a plurality of parameter values (for example, a plurality of window widths) that could be specified, which enhances the user's convenience.

Next, determining the range to generate ground-truth data S308 and generating ground-truth data S309 are performed. A time period including a start time and an end time with the name of a working activity associated with this period is determined to be the range to generate ground-truth data for the activity recognition result displayed by the ground-truth data input and output program 22. Examples of determining the range 60 include determining a range automatically selected in the descending order of the probability of work and determining a range that is specified by the user from the displayed activity recognition result.

When a range to generate ground-truth data is determined (S308), statistics information on the unit activity series data 47 in the determined ground-truth data generation range and appropriateness of the recognition result in the determined ground-truth data generation range are displayed. The statistics information can be the frequencies of unit activities or the order of unit activities. The appropriateness of the recognition result can be the probabilities of works. The user inputs whether the recognition result is correct or not and if wrong, a correction to the displayed information. Since the unit activities are provided with interpretable information on motions, the user can determine what to input based on the information on the motions included in the ground-truth data generation range.

As soon as the user inputs confirmation on the recognition result in the specified ground-truth data generation range as described above, the working activity in the time period is recorded to the working activity ground-truth data 44 and the ground-truth is fixed (61). Determining a range to generate ground-truth data S308 and generating ground-truth data S309 can be repeated as many times as the user wants.

At the end, updating the working activity model S310 is performed using the obtained working activity ground-truth data 44, unit activity series data 47, and learning range data 48. This updating the working activity model S310 does not need to be performed each time generating ground-truth data S309 is completed. For example, updating the working activity model S310 can be performed when ground-truth data is accumulated into an amount satisfying a predetermined condition, or when ground-truth data about a user satisfying a predetermined condition (such as a new user or a user belonging to a new field) is accumulated.

Through the above-described processing to generate ground-truth data S301, working activity ground-truth data 44 can be easily generated only from sensor data 41 in which human activities are recorded. Since the accuracy of the information displayed for the user improves as the working activity model 45 is updated with the generated working activity ground-truth data 44, the user can create working activity ground-truth data 44 more smoothly by continuously using this ground-truth data creation support system.

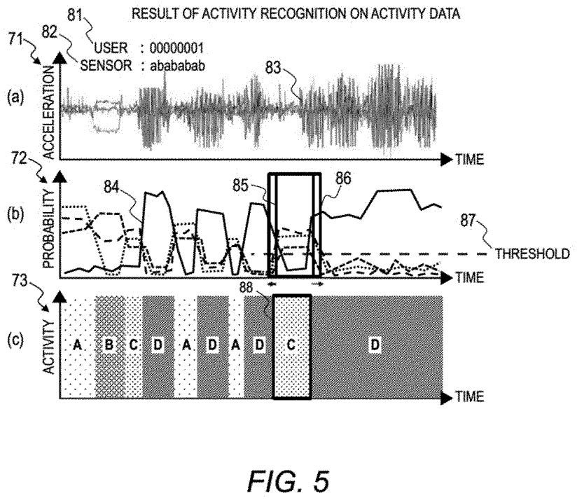

FIG. 5 is a schematic diagram illustrating data forms in obtaining ground-truth candidate data 49 from sensor data 41 using the ground-truth data creation support system in Embodiment 1 of this invention.

The graph 71 (FIG. 5(a)) is an example of displayed acceleration data in the sensor data 41 received by the server 5 from the sensor 1. The received acceleration data is time-series data including user information 81 and sensor information 82; the graph 71 shows three kinds of acceleration data 83 along three axes orthogonal to one another.

The graph 72 (FIG. 5(b)) is an example of displayed ground-truth candidate data 49 obtained by converting the acceleration data 83 into unit activity series data 47 with a unit activity model 43 and further converting the acquired unit activity series data 47 with a working activity model 45. The ground-truth candidate data 49 is time-series probability data 84 on works.

The graph 73 (FIG. 5(c)) is an example of displayed ground-truth candidate data 49 calculated from the work probability data 84 in the graph 72 out of the data included in ground-truth candidate data 49. Typically, the working activity in the ground-truth candidate data is defined as the working activity 88 ranked the top (or having the highest probability) at each time. A working activity in a given time period can be calculated based on the time period (for example, a selected section 85) in which the working activity calculated at each time is continued.

Before and after a working activity changes to another, a plurality of activities could be made in parallel. In this case, the time period for ground-truth candidate data 49 could be separated to shorter ones. To prevent this situation, instead of simply employing the time period in which the working activity with the highest probability continues as the section 85 for the ground-truth candidate data 49, a threshold 87 for the time period of the same working activity can be defined at, for example a half value of the highest probability. The time period in which the same working activity keeps showing a probability equal to or higher than this threshold 87 can be employed as the section 86 for the ground-truth candidate data 49, for example in calculating the appropriateness of the recognition result to be displayed in response to the processing S308 of determining a range to generate ground-truth data in the ground-truth data input and output program 22. Alternatively, a threshold to employ the working activity can be defined and if the probability of a work is lower than this threshold, the ground-truth candidate data 49 at the time does not need to be calculated.

FIG. 6 is an explanatory diagram illustrating an example of an input screen to receive confirmation of a ground-truth 61 from the operator with the ground-truth data creation support system in Embodiment 1 of this invention.

This input screen is displayed by the ground-truth data input and output program 22 on the PC 2 or smartphone 3. The band chart 73 in the lower tier is the graph 73 in FIG. 5(c), which is time-series ground-truth candidate data displayed as a result of activity recognition, and shows a working activity 95 (for example, eating assistance) as a candidate for the ground-truth in each section (for example, in the section 85).

The knob for the unit activity window width displayed in the region 94 is operated to change the hyperparameter in obtaining unit activity series data 47 that is used to acquire the ground-truth candidate data 49 from the sensor data 41. FIG. 6 shows an example where the unit activity window width as one of the hyperparameters is variable. The user can specify a unit activity window width by operating the unit activity window width knob in the region 94. This operation to specify a unit activity window width corresponds to selecting a unit activity model employing the specified unit activity window width from a plurality of prepared unit activity models and a working activity model associated with the selected unit activity model.

If unit activity series data 47 is calculated in advance using a number of unit activity models having different values in a certain range for the unit activity window width, the user can instantly acquire corresponding unit activity series data 47 when the user moves the unit activity window width knob within the range. That is to say, pseudo real-time operation is available. A value outer than the range can also be specified although calculation may take time. The unit activity series data 47 is recalculated and displayed after the value is specified. In the case where the unit activity model 43 and the working activity model 45 include hyperparameters other than the window width, the input screen can provide selections about each hyperparameter.

The frame in the middle of the lower tier represents a section (selected section) 85 specified for a range to generate ground-truth data. This selected section is an example of the specific measurement time period described with reference to FIG. 1.

The region 90 shows unit activity series data 47 in the selected section 85. This data 47 shows unit activity IDs at individual times in the selected section 85 or the variation in unit activity ID with time.

The region 91 shows the proportions of unit activity series data in the selected section 85. Although this example shows the proportions of unit activity series data 47, this region 91 can show the frequencies using a histogram, for example. As shown in the region 91, each unit activity is provided with a unit activity ID (for example, 0) and a unit activity name (for example, slow movement).

The region 92 shows examples of one or more kinds of sensor data 41 classified as some unit activity.

The region 93 shows the appropriateness of the recognition result about the working activity name in the selected section 85. Typically, a recognition result in the region 93 shows the names of working activities in the descending order of probability output by the working activity model 45.

As to the regions 90 to 93, all of them can be displayed or alternatively, one or more of them can be displayed as necessary.

The region 96 shows the start time and the end time of the selected section 85 and a working activity field 95. The working activity field 95 shows the name of the working activity with the highest probability in the selected section 85 (in the example of FIG. 6, “C: eating assistance”). This corresponds to the ground-truth candidate data 49 in the selected section 85.

The user determines whether the working activity displayed in the field 95 matches the working activity actually performed in the selected section 85 (whether the ground-truth candidate data 49 in the selected section 85 is correct) with reference to the region 96. At this time, the user can check the unit activities in the selected section 85 and the representative sensor data for each unit activity displayed in the regions 90 to 92, in addition to the user's own memory, to determine whether the ground-truth candidate data 49 in the selected section 85 is correct. The user can also check some working activities with high probabilities shown in the region 93 against his/her own memory to determine whether the ground-truth candidate data 49 in the selected section 85 is correct and further determine the true ground-truth if the ground-truth candidate data 49 is wrong.

The user can input affirmation in the case where the ground-truth candidate data 49 in the selected section 85 is correct, and correction in the case where it is wrong. Such input of affirmation or correction corresponds to input of the correct working activity. This input is made by the user operating the PC 2 or smartphone 3, which is a part of the function of the input unit 1001 in FIG. 1. If the ground-truth candidate data 49 is affirmed, the ground-truth candidate data 49 is stored in the working activity ground-truth data 44. If correction is input, the input working activity is stored in the working activity ground-truth data 44.

As understood from the above, when a user is going to input which working activity is performed in the selected section 85, the ground-truth data creation support system makes the user recall the memory when sensor data 41 is measured by presenting not only ground-truth candidate data 49 but also statistical information on human-interpretable unit activities in the region 90 and 91. Therefore, the user can input whether the ground-truth candidate data 49 is correct or wrong to store correct working activity ground-truth data 44 with reference to quantitative information.

In FIG. 6, information on unit activities based on unit activity series data 47 in a selected section converted by a unit activity model 43 including one hyperparameter is presented. However, the ground-truth input and output program 22 can present information on unit activities based on a plurality of sets of unit activity series data 47 converted by a plurality of unit activity models 43 different in hyperparameter.

For example, the program 22 can display information on unit activities obtained by changing the unit activity window of a hyperparameter (for example, 6 seconds) into a plurality of different values (for example, 2 seconds, 6 seconds, and 15 seconds). Further, in addition to displaying information on unit activities converted by a plurality of unit activity models 43 including different hyperparameters, the program 22 can display a plurality of sets of ground-truth candidate data converted by a plurality of working activity models 45 including different hyperparameters.

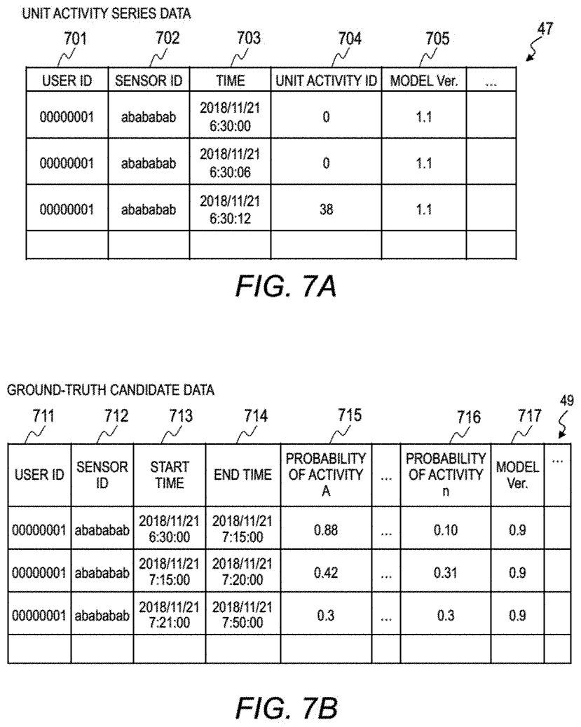

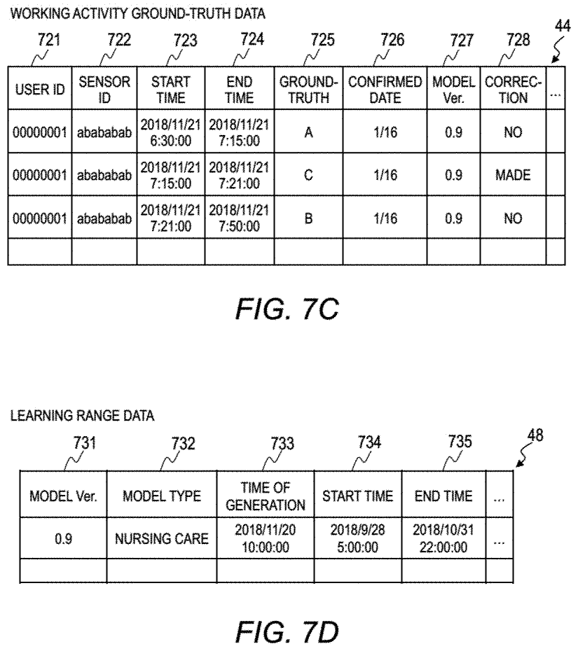

FIG. 7A is an explanatory diagram of a typical example of data structure of unit activity series data 47 stored in the ground-truth data creation support system in Embodiment 1 of this invention.

FIG. 7B is an explanatory diagram of a typical example of data structure of ground-truth candidate data 49 stored in the ground-truth data creation support system in Embodiment 1 of this invention.

FIG. 7C is an explanatory diagram of a typical example of data structure of working activity ground-truth data 44 stored in the ground-truth data creation support system in Embodiment 1 of this invention.

FIG. 7D is an explanatory diagram of a typical example of data structure of learning range data 48 stored in the ground-truth data creation support system in Embodiment 1 of this invention.

The unit activity series data 47 (FIG. 7A) typically includes information of a user ID 701, a sensor ID 702, a time 703, a unit activity ID 704, and model information 705 in one record.

The user ID 701 and the sensor ID 702 are identification information on a user (or the person wearing a sensor 1) and identification information on the sensor 1, respectively. The time 703 is the time of acquisition of the sensor data used to recognize a unit activity ID (for example, in the case where a unit activity ID is calculated from sensor data in a period of six seconds, the start time of the period). The unit activity ID 704 is the calculated unit activity ID and the model information 705 is identification information (such as a version number) on the unit activity model 43 used to calculate the unit activity ID.

The ground-truth candidate data 49 (FIG. 7B) typically includes a user ID 711, a sensor ID 712, a start time 713 of the duration of the same working activity, an end time 714 of the duration of the same working activity, average probabilities 715 to 716 of working activities in the section, and model information 717 in one record.

The user ID 711 and the sensor ID 712 are the same as the user ID 701 and the sensor ID 702 in the unit activity series data 47. The start time 713 and the end time 714 of the duration of the same working activity 713 are the start point and the end point of the time period in which the same working activity is inferred to be continued. These can be the start point and the end point of the selected section 85 shown in FIGS. 5 and 6. This section can be the section 86 for ground-truth candidate data shown in FIG. 5.

The average probabilities 715 to 716 of the working activities in the section are the probabilities of the working activities recognized in the section. Although FIG. 7B shows the probability 715 of the working activity A and the probability 716 of the working activity n by way of example and omits the other probabilities, the probabilities of any number n of working activities such as a working activity B, and a working activity C are recorded in the actual cases. The model information 717 is identification information (such as a version number) on the working activity model 45 used to infer those working activities (or used to generate the ground-truth candidates).

Instead of the start time 713 and the end time 714 of the duration of the same working activity and the average probabilities 715 to 716 of working activities in the section, times with intervals of a window width at which working activity recognition is performed and the probabilities of working activities at each of the times can be recorded in the ground-truth candidate data 49.

Particularly, this embodiment is supposed to use a plurality of unit activity models 43 different in hyperparameter to obtain unit activity series data 47 and further, to use a plurality of working activity models 45 different in hyperparameter to calculate ground-truth candidate data 49. Accordingly, it is preferable that each record include model information indicating which model is used to generate the record. Then, the user can compare recognition results before and after the hyperparameter is changed to readily find a hyperparameter suitable for the working activity the user wants to be recognized.

The working activity ground-truth data 44 (FIG. 7C) typically includes a user ID 721, a sensor ID 722, a start time 723, an end time 724, a ground-truth 725, a working activity confirmed date 726, model information 727, and correction to ground-truth candidate data 728 in one record.

The user ID 721 and the sensor ID 722 are the same as the user ID 701 and the sensor ID 702 in the unit activity series data 47. The start time 723 and the end time 724 are the same as the start time 713 and the end time 714 of the duration of the same working activity in the ground-truth candidate data 49. The ground-truth 725 is the name of the correct working activity confirmed by the user and the working activity confirmed date 726 is the date on which the working activity is confirmed. The model information 727 is identification information (such as a version number) on the working activity model 45 used to calculate a ground-truth candidate and the correction to ground-truth candidate data 728 indicates whether the ground-truth candidate is corrected with the working activity name provided by the user. The value “NO” in the correction to the ground-truth candidate data 728 means that the ground-truth candidate is not changed, or that the ground-truth candidate (the working activity with the highest probability) is the ground-truth 725.

The correction to ground-truth candidate data 728 is not requisite for the working activity ground-truth data 44 but preferably, it is to be included in consideration of the possibility to evaluate the accuracy in recognition of the working activity model 45. This embodiment is based on an assumption that the user of the sensor 1 is the same person as the creator of the working activity ground-truth data 44; however, if the creator of the working activity ground-truth data 44 is different from the user like in the case where the user's supervisor creates the working activity ground-truth data 44, it is preferable to record the ID of the user who confirms the working activity together.

The learning range data 48 (FIG. 7D) typically includes model information 731, a model type 732, a time of generation 733, and a start time 734 and an end time 735 of the learning range in one record. The model information 731 is identification information (such as a version number) of a generated working activity model 45 and corresponds to the model information 717. The model type 732 indicates the type of the working activity model 45. Particularly about the working activity model 45, the works to be recognized are expected to be different significantly depending on its application field and therefore, it is preferable that the model type depending on the field (such as nursing care or construction) of the person who specifies the learning range be recorded.

The time of generation 733 is a time at which the working activity model 45 is generated. The start time 734 and the end time 735 of the learning range are the times at the start point and the end point of the data used to generate the working activity model 45.

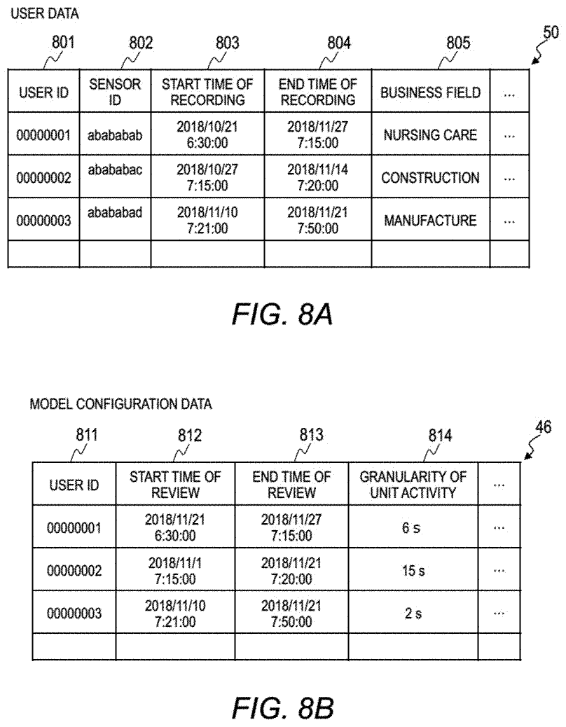

FIG. 8A is an explanatory diagram of a typical example of data structure of the user data 50 held by the ground-truth data creation support system in Embodiment 1 of this invention.

FIG. 8B is an explanatory diagram of a typical example of data structure of the model configuration data 46 held by the ground-truth data creation support system in Embodiment 1 of this invention.

The user data 50 (FIG. 8A) typically includes a user ID 801, a sensor ID 802, and a start time of recording 803, an end time of recording 804, and business field information 805 in one record.

The user ID 801 and the sensor ID 802 are the same as the user ID 701 and the sensor ID 702 in the unit activity series data 47. The start time 803 and the end time 804 of recording are the dates and times of the start point and the end point of recording sensor data on the user. The business field information 805 is information indicating the business field the user belongs to. It is desirable that a model type 732 associated therewith be configured.

In addition to the foregoing information, the user data 50 may hold information such as a duration of service and/or a job type of the user, depending on the analysis policies for the collected data.

The model configuration data 46 (FIG. 8B) typically includes a user ID 811, a start time 812 and an end time 813 of review, and hyperparameters (such as a unit activity window width) included in the model in one record. The user ID 811 is the same as the user ID 701 in the unit activity series data 47. The start time 812 and the end time 813 of review are the times of the start point and the end point of the data used to generate the working activity model 45. The granularity of unit activity 814 is an example of a hyperparameter included in the model and indicates a unit activity window width (for example, 2 seconds, 6 seconds, or 15 seconds).

This embodiment is described based on an assumption that the unit activity model 43 and the working activity model 45 include only a unit activity window width as the hyperparameter. However, in the case where more hyperparameters (such as the number of clusters for the unit activity model 43) are included, the model configuration data 46 can store all of the information or only information operable by the user.

The above-described system in Embodiment 1 assigns human-interpretable codes, namely unit activities (unit activity series data 47), to sensor data 41, so that the user can understand the recognized working activity (ground-truth candidate data 49) is composed of what unit activities. Accordingly, the system can support the user in creating accurate ground-truth data. In addition, the user can determine whether ground-truth candidate data 49 is correct with reference to the unit activity series data 47 and therefore, even if the user is different from the user wearing the sensor, the user can create ground-truth data. Further, the system presents unit activity series data 47 constituting ground-truth candidate data 49 and statistical information on the unit activity series data 47 to the user, so that the user can have more information to determine working activity ground-truth data 44. The system can support the user in creating more accurate ground-truth data. In addition, the system allows quantitative comparison of the differences among a plurality of working activities with unit activity series data 47, which is achieved by comparing different ground-truth candidate data 49 for the same working activity or different working activities with the unit activity series data 47 constituting those ground-truth candidate data 49.

### Embodiment 2

Hereinafter, Embodiment 2 of this invention is described. Except for the differences described in the following, each unit in the ground-truth data creation support system in Embodiment 2 has the same function as the unit assigned the same reference sign in Embodiment 1; the descriptions thereof are omitted here.

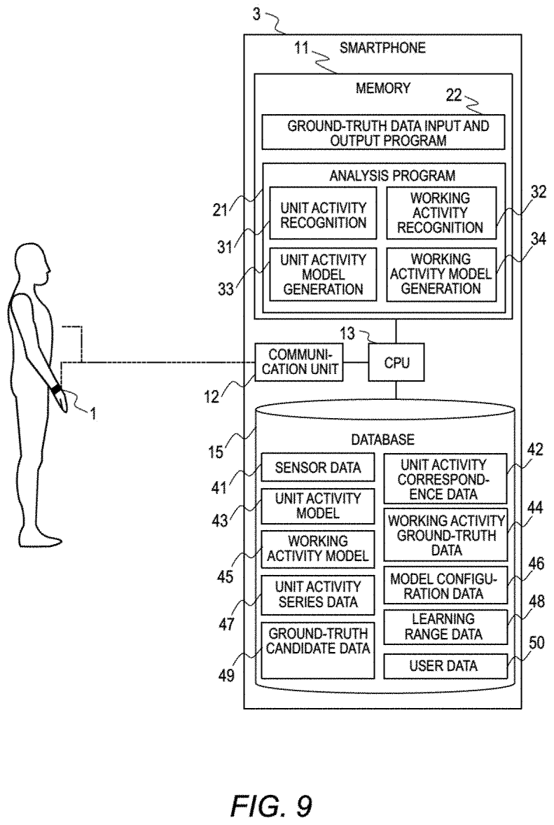

FIG. 9 is a hardware configuration diagram of the ground-truth data creation support system in Embodiment 2 of this invention.

In Embodiment 2, the PC2 or smartphone 3 executes all processing of the analysis program performed by the server 5 in Embodiment 1. Alternatively, the sensor 1 can execute a part of the processing of the analysis program performed by the server 5 in Embodiment 1 and the PC 2 or smartphone 3 can execute the remaining processing. FIG. 9 illustrates a configuration of the ground-truth data creation support system in the case where the smartphone 3 executes all processing of the analysis program, by way of example.

Embodiment 2 analyzes sensor data 41 measured by the sensor 1 without sending it to the server 5 via the network 4 and therefore, has advantages such as good responsivity and less communication traffic, in addition to the advantages of Embodiment 1.

### Embodiment 3

Hereinafter, Embodiment 3 of this invention is described. Except for the differences described in the following, each unit in the ground-truth data creation support system in Embodiment 3 has the same function as the unit assigned the same reference sign in Embodiment 1; the descriptions thereof are omitted here.

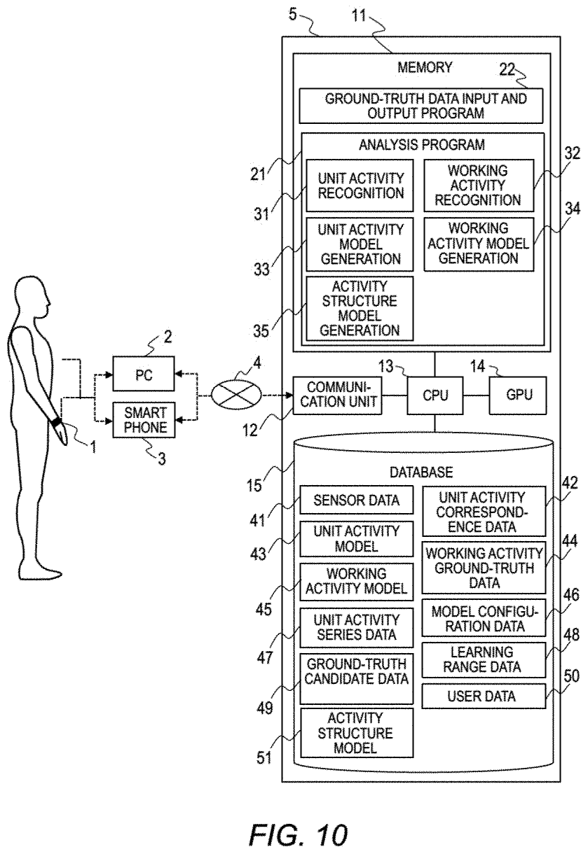

FIG. 10 is a hardware configuration diagram illustrating a major configuration of the ground-truth data creation support system in Embodiment 3 of this invention.

In recording working activity ground-truth data 44 obtained through confirmation of a ground-truth 61, the server 5 in Embodiment 3 records the working activity in a selected range together with the unit activities included in the selected range to the working activity ground-truth data 44 in a form that can hold their parent-child relation, such as a tree structure or a graph structure. The server 5 subsequently executes an activity structure model generation program 35 to learn the parent-child relation between the unit activities and the working activity with a known structured learning algorithm and holds the relation in an activity structure model 51.

Hereinafter, among activities having a parent-child relation, the activity corresponding to a parent is referred to as higher-level activity, an activity corresponding to a child is referred to as lower-level activity. Taking an example where a working activity is recognized for some time period, a parent-child relation such that the working activity is a higher-level activity and the unit activities included in the time period are lower-level activities is established.

The parent-child relation in this embodiment can include not only an example that unit activities are lower-level activities and a working activity is a higher-level activity but also an example that a working activity is a lower-level activity and another working activity is a higher-level activity. Taking an example of the nursing care field, a shift work can be a higher-level work activity in relation to a time period including eating assistance and moving assistance as lower-level working activities. That is to say, the working activity model 45 in this embodiment includes not only a model for recognizing a working activity based on unit activities but also a model for recognizing a higher-level working activity based on lower-level working activities.

In the subsequent phase of presenting information on unit activities in a selected range as illustrated in FIG. 6, the server 5 presents the working activity in the selected range and the unit activities included in the selected range in the form such that the user can understand the parent-child relation, for example in a tree structure or a graph structure calculated by the activity structure model 51, in place of or together with the information provided in FIG. 6.

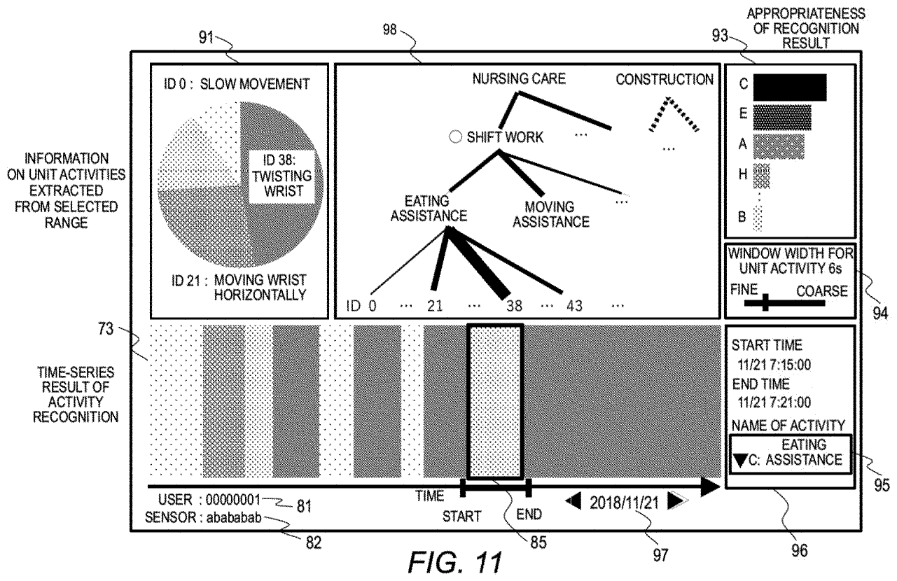

FIG. 11 is an example of an input screen to receive confirmation of a ground-truth 61 from the operator with the ground-truth data creation support system in Embodiment 3 of this invention.

The region 98 is an example where an activity structure (or a hierarchical structure of activities) about the working activity in a selected range. Embodiment 3 presents a typical unit activity pattern included in the working activity in the selected range in a tree structure calculated by the activity structure model 51 together in presenting information on unit activities in the selected range.

Each node of the tree structure represents a working activity in each level in the case where the working activities have a parent-child relation (in other words, the working activities have a hierarchical structure). The nodes of the lowermost level represent unit activity IDs. The thickness of each edge represents a typical composition rate (for example, a rate of the frequency or a rate of the time length of appearance) of the lower-level activity in the higher-level activity. Further, the region 98 can show a tree structure of the working activities in the selected range in the case where a working activity model 45 for a different application field is used.

The foregoing Embodiment 3 provides the basis of recognition of a working activity in each time period and therefore, in addition to the advantages same as those in Embodiment 1, Embodiment 3 supports the user more effectively in creating accurate ground-truth data on activities.

It should be noted that this invention is not limited to the above-described embodiments but include various modifications. For example, the above-described embodiments provide details for the sake of better understanding of this invention; they are not limited to those including all the configurations as described. A part of the configuration of an embodiment may be replaced with a configuration of another embodiment or a configuration of an embodiment may be incorporated to a configuration of another embodiment. A part of the configuration of an embodiment may be added, deleted, or replaced by that of a different configuration. The above-described configurations, functions, processing units, and processing means, for all or a part of them, may be implemented by hardware: for example, by designing an integrated circuit. The above-described configurations and functions may be implemented by software, which means that a processor interprets and executes programs providing the functions. The information of programs, tables, and files to implement the functions may be stored in a storage device such as a memory, a hard disk drive, or an SSD (Solid State Drive), or a computer-readable non-transitory data storage medium such as an IC card, an SD card, or a DVD.

The drawings include control lines and information lines as considered necessary to explain the embodiments but do not include all control lines or information lines in the actual products to which this invention is applied. It can be considered that most of all components are interconnected.

What is claimed is:

1. A ground-truth data creation support system comprising:
an input unit configured to input sensor data obtained by measurement with a sensor;
a storage unit configured to store a code assigning model to assign codes associated with characteristics of sensor data to the sensor data and an activity inferring model to infer an activity of a person wearing the sensor based on the sensor data that has been assigned codes;
a processing unit configured to infer an activity in a specific measurement time period of a person wearing the sensor, based on sensor data in the specific measurement time period, the code assigning model, and the activity inferring model; and
an output unit configured to output the inferred activity in the specific measurement time period and codes assigned to the sensor data in the specific measurement time period.

2. The ground-truth data creation support system according to claim 1, further comprising:
a display unit configured to display the inferred activity in the specific measurement time period and information on the sensor data in the specific measurement time period that has been assigned codes.

3. The ground-truth data creation support system according to claim 2,
wherein the input unit is configured to input a ground-truth of the activity in the specific measurement time period, and
wherein the storage unit is configured to store the ground-truth of the activity in the specific measurement time period.

4. The ground-truth data creation support system according to claim 3,
wherein the storage unit is configured to store a plurality of code assigning models and a plurality of activity inferring models,
wherein the processing unit is configured to obtain a plurality of inference results about the activity in the specific measurement time period of the person wearing the sensor based on the plurality of code assigning models and the plurality of activity inferring models,
wherein the input unit is configured to receive selection of a code assigning model and an activity inferring model, and
wherein the display unit is configured to display an inference result based on the selected code assigning model and the selected activity inferring model out of the plurality of inference results.

5. The ground-truth data creation support system according to claim 4,
wherein the plurality of code assigning models include a plurality of models each configured to assign codes associated with characteristics of sensor data to the sensor data based on a different parameter,
wherein the input unit is configured to input a parameter as the selection of a code assigning model and an activity inferring model, and
wherein the display unit is configured to display an inference result based on a code assigning model associated with the input parameter and an activity inferring model associated with the code assigning model out of the plurality of inference results.

6. The ground-truth data creation support system according to claim 3,
wherein the activity inferring model includes a model configured to infer lower-level activities of the person wearing the sensor based on the sensor data that has been assigned codes and a model configured to infer a higher-level activity of the person wearing the sensor based on the lower-level activities,
wherein the processing unit is configured to infer lower-level activities and a higher-level activity in the specific measurement time period of the person wearing the sensor, and
wherein the display unit is configured to display information indicating a hierarchical structure of the inferred activities.

7. The ground-truth data creation support system according to claim 3, wherein the processing unit is configured to:
generate an activity inferring model based on the sensor data in the specific measurement time period and a ground-truth of the activity in the specific measurement time period; and
update the activity inferring model stored in the storage unit with the generated activity inferring model.

8. The ground-truth data creation support system according to claim 2, wherein the display unit is configured to display at least one of proportions of the codes assigned to the sensor data in the specific measurement time period, the codes assigned to the sensor data at individual times in the specific measurement time period, one or more sensor data in the specific measurement time period that has been assigned a code, and appropriateness of an inferred activity in the specific measurement time period as the information on the sensor data in the specific measurement time period that has been assigned codes.

9. The ground-truth data creation support system according to claim 1, wherein the sensor data is acceleration data measured by an acceleration sensor.

10. The ground-truth data creation support system according to claim 1, wherein the code assigning model is a model configured to cluster the sensor data based on feature values and assign cluster identifiers as the codes.

11. The ground-truth data creation support system according to claim 1, wherein the processing unit is configured to:
generate a code assigning model based on sensor data in a case where a predetermined condition is satisfied; and
update the code assigning model stored in the storage unit with the generated code assigning model.

12. The ground-truth data creation support system according to claim 11, wherein the predetermined condition is at least either one of a condition that a predetermined amount of new sensor data is input and a condition that sensor data on a new person is input.

13. The ground-truth data creation support system according to claim 1, wherein the activity inferring model is a model configured to infer an activity of a person wearing the sensor based on frequency of appearance of the codes assigned to the sensor data in the specific measurement time period.

14. A ground-truth data creation support method comprising:
an input step of inputting sensor data obtained by measurement with a sensor;
a storing step of storing a code assigning model to assign codes associated with characteristics of sensor data to the sensor data and an activity inferring model to infer an activity of a person wearing the sensor based on the sensor data that has been assigned codes;
a processing step of inferring an activity in a specific measurement time period of the person wearing the sensor, based on sensor data in the specific measurement time period, the code assigning model, and the activity inferring model; and
an output step of outputting the inferred activity in the specific measurement time period and codes assigned to the sensor data in the specific measurement time period.
